(19) United States
(12) Patent Application Publication
(10) Pub. No.: US 2024/0301021 A1
(43) Pub. Date: Sep. 12, 2024

(54) ENHANCER SEQUENCES SPECIFIC FOR CARDIAC FIBROBLASTS AND METHODS OF USE THEREOF

(71) Applicants: Tenaya Therapeutics, Inc., South San Francisco, CA (US); The J.David Gladstone Institutes, A Testamentary Trust Established Under the Will of J.David, San Francisco, CA (US)

(72) Inventors: Laura LOMBARDI, Redwood City, CA (US); Katherene S. POLLARD, San Francisco, CA (US); Sean H. WHALEN, San Francisco, CA (US)

(21) Appl. No.: 18/547,843

(22) PCT Filed: Feb. 24, 2022
(86) PCT No.: PCT/US2022/017709

§ 371 Date: Aug. 24, 2023

Related U.S. Application Data
(60) Provisional application No. 63/153,532, filed on Feb. 25, 2021.

(51) Int. Cl. C07K 14/47; C12N 15/86

(52) U.S. Cl. CPC C07K 14/4705; C12N 15/86; C12N 2740/15043; C12N 2830/008

(57) ABSTRACT

The disclosure provides cardiac cell-specific enhancer sequences. The enhancers are specific to cardiac cells, such as cardiac fibroblasts. Also provided are polynucleotides, vectors, cells, compositions and kits, and methods of use thereof.

### CROSS REFERENCE TO RELATED APPLICATIONS

This application claims the benefit of priority to U.S. Provisional Patent Application Ser. No. 63/153,532 filed on Feb. 25, 2021, the contents of which are hereby incorporated by reference in their entireties.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is TENA_026_01 WO_ST25.txt. The text file is about 10 KB, created on Feb. 24, 2022, and is being submitted electronically via EFS-Web.

### BACKGROUND

The expression level of exogenous gene products in cells relies on functional polynucleotide regulatory elements. The functional regulatory elements, or transcriptional regulatory elements, can include, for example, promoter, activator, and/or enhancer polynucleotide sequences. The transcriptional regulatory elements (TREs) can be linked to a polynucleotide sequence encoding an exogenous gene product and delivered to a target cell in a vector, such as a plasmid or viral vector.

TREs that can control expression of exogenous gene products in a tissue or cell-specific manner have many advantages over TREs that promote expression of exogenous gene products in any tissue or cell type. Expression of an exogenous gene product may have desirable effects in one cell type and undesirable effects in another cell type, so delivery of an expression vector to a tissue of multiple cell types may have negative off-target effects. In some applications it is desirable to target a specific cell type within a tissue to express an exogenous gene product that influences cell fate determination, such as targeted differentiation or apoptosis. For example, targeted expression of exogenous gene products in cardiac fibroblasts can be used to direct differentiation toward a cardiomyocyte phenotype. This kind of cell type-specific expression of transgenes encoding exogenous gene products can be useful for in vitro and in vivo cardiac research and therapeutic applications. However, cardiac fibroblast-specific TREs, such as enhancers, are not readily available for use in vectors to express exogenous gene products.

There remains a need in the art for improved cardiac fibroblast-specific enhancer sequences and methods of use.

### SUMMARY

The present invention relates generally to enhancer sequences for gene expression is a cell-type specific manner, as well compositions and methods of use thereof.

In one aspect, the disclosure provides a recombinant polynucleotide, comprising a cardiac cell-specific enhancer that shares at least 80% identity to any one of SEQ ID NOs: 1-21. In some embodiments, the polynucleotide comprises a sequence encoding a transgene product operatively linked to the enhancer. In some embodiments, the cardiac cell-specific enhancer is a cardiac fibroblast-specific enhancer. In some embodiments, the polynucleotide expresses the transgene product at a level and/or rate of at least two, at least three, at least four, at least five, at least six, at least seven, at least eight, at least nine, or at least ten times greater in cardiac fibroblasts than cardiomyocytes. In some embodiments, the polynucleotide expresses the transgene product at a level and/or rate of at least two times greater in cardiac fibroblasts than cardiomyocytes. In some embodiments, the polynucleotide comprises a sequence that shares at least 80%, at least 85%, at least 90%, at least 95%, at least 98%, or 100% identity to SEQ ID NOs: 1-21. In some embodiments, the polynucleotide comprises a sequence that shares at least 80%, at least 85%, at least 90%, at least 95%, at least 98%, or 100% identity to at least two sequences selected from SEQ ID NOs: 1-21. In some embodiments, the polynucleotide comprises a sequence that shares at least 80%, at least 85%, at least 90%, at least 95%, at least 98%, or 100% identity to SEQ ID NOs: 19. In some embodiments, the polynucleotide comprises a sequence that shares at least 80%, at least 85%, at least 90%, at least 95%, at least 98%, or 100% identity to SEQ ID NOs: 20. In some embodiments, the polynucleotide comprises a sequence that shares at least 80%, at least 85%, at least 90%, at least 95%, at least 98%, or 100% identity to SEQ ID NOs: 21.

In one aspect, the disclosure provides a vector comprising a polynucleotide described herein. In some embodiments, vector is a viral vector. In some embodiments, the viral vector is a lentivirus, a retrovirus, an adenovirus, an adeno-associated virus, or a hybrid virus. In some embodiments, the vector is a plasmid. In some embodiments, the vector is an artificial chromosome.

In one aspect, the disclosure provides an isolated cell comprising a polynucleotide described herein. In another aspect, the disclosure provides an isolated cell comprising a vector described herein. In some embodiments, the cell is a cardiac cell. In some embodiments, the cell is a cardiac fibroblast and/or cardiomyocyte. In some embodiments, the cell is a stem cell. In some embodiments, the cell is an induced pluripotent stem cell.

In one aspect, the disclosure provides a pharmaceutical composition comprising a cell described herein.

In one aspect, the disclosure provides a method of expressing a transgene product, comprising introducing a polynucleotide into a cell, wherein the polynucleotide comprises a cell-specific enhancer sequence described herein. In some embodiments, the polynucleotide comprises a sequence encoding a transgene product operably linked to the enhancer. In some embodiments, the polynucleotide is introduced into the cell using a vector described herein. In some embodiments, the cell is a cardiac cell. In some embodiments, the cell is a cardiac fibroblast and/or cardiomyocyte. In some embodiments, the cell is a stem cell. In some embodiments, the cell is an induced pluripotent stem cell. In some embodiments, the transgene product is expressed at a level and/or rate of at least two, at least three, at least four, at least five, at least six, at least seven, at least eight, at least nine, or at least ten times greater in cardiac fibroblasts than cardiomyocytes.

Other features and advantages of the disclosure will be apparent from and encompassed by the following detailed description and claims.

### BRIEF DESCRIPTIONS OF DRAWINGS

FIG. 1 is a diagram depicting a computational process for generating putative enhancer sequences from two data sets. A series of filters is performed on candidate enhancer regions, wherein the filters identify sequences based on experimental data determining histone modifications (H3 K4Me3, H3 K27Ac) using chromatin immunoprecipitation (ChIP) and sequence comparisons (e.g. NOT overlapping). The filters are designed to identify putative enhancer sequences specific to human cardiac fibroblasts (HCFs) and not cardiomyocytes (CMs), embryonic stem cell cardiomyocytes (ES-CMs), induced pluripotent stem cell derived cardiomyocytes (iPSC-CMs), and human umbilical vein endothelial cells (HUVECs).

FIG. 2 is a diagram illustrating the experimental workflow for testing putative enhancer sequences in a primary screen. Also depicted illustrative expression constructs used in the primary screen. The constructs contain the putative enhancer sequence operably linked to a minimal promoter (mP) and a reporter gene (GFP).

FIG. 3 is a table and plot showing enrichment of putative sequences in a primary screen of enhancer sequences in cells sorted by FACS. The table shows the number of shared enrichment regions identified that are implicated as putative enhancer sequences with human cardiac fibroblast specificity. The plot shows the overlap in putative enhancer-mediated expression of a reporter gene between two human cardiac fibroblast cell lines.

FIG. 4 is a diagram depicting the experimental design for a secondary screen to validate putative human cardiac fibroblast specific enhancer sequences. Combinations of enhancer sequences are operably linked to a minimal promoter or a super core promoter and a reporter gene and packaged into a lentiviral vector Human cardiac fibroblasts (HCFs) or induced pluripotent stem cell derived cardiomyocytes (iPSC-CMs) are infected with the lentiviral vectors and analyzed by flow cytometry.

FIG. 5 is a series of plots showing experimental validation and selectivity of putative enhancer sequences. A) is a plot showing that many of the tested putative enhancer sequences operably linked to a minimal promoter (mP) and reporter gene show high expression in human cardiac fibroblasts (HCFs) compared to mP only. B) is a plot showing the expression level of a reporter gene operably linked to putative enhancer sequences and mP in either HCFs or induced pluripotent stem cell derived cardiomyocytes (iPSC-CMs). Expression levels are normalized to expression of the reporter gene operably linked to mP only. C) is a plot showing the expression levels of a reporter gene operably linked to enhancer sequences and either a super core promoter (SCP) or mP in human cardiac fibroblasts (hCF). The expression levels are normalized to reporter gene expression levels operably linked to the same enhancer/promoter combination in cardiomyocytes.

### DETAILED DESCRIPTION

### Overview

The disclosure relates generally to enhancer sequences for selectively expressing exogenous gene products (transgenes) specifically in cardiac fibroblasts. The disclosure provides enhancer sequences, polynucleotides and vectors comprising the enhancer sequences and enhancer sequences operably linked to one or more gene or nucleotide sequences encoding a gene product (i.e., transgenes), cells comprising the polynucleotides, vectors, and enhancer sequences described herein, compositions and kits comprising the enhancer sequences, polynucleotides, vectors, and cells described herein, and methods of use thereof.

The enhancer sequences, polynucleotides, vectors, cells, compositions and kits, and methods of use provided in the disclosure are useful in applications where it is desirable to express a gene or nucleotide sequence encoding a gene product, i.e. a transgene, specifically in a cardiac cell, e.g. a cardiac fibroblast. Applications can include, for example, biomedical research and therapeutic approaches. Biomedical research applications can be concerned with, for example, determining the effects of a gene product's expression in a cardiac-specific cell on biological function. Biological function can include, for example, intracellular effects, intercellular effects, effects on specific cellular phenotypes, effects on tissue (e.g. cardiac tissue), effects on whole organs (e.g. the heart), and/or effects on disease phenotype (e.g. in an organism). Biological function can be assessed using the enhancer sequences provided both in vitro and in vivo. Therapeutic approaches can include, for example, enhancer sequences of the disclosure used in gene therapies. Gene therapies can be any therapeutic approach that seeks to modify or manipulate the expression of a gene or sequence encoding a gene product, and/or to alter the biological properties of living cells for therapeutic use. Gene therapies include all therapies that mediate their effects by transcription or translation of transferred genetic material or by specifically altering host genetic sequences. Some examples of gene therapy products include polynucleotides (e.g., plasmids, in vitro transcribed ribonucleic acid (RNA)), engineered viruses (e.g., lentivirus and adeno-associated virus), engineered site-specific nucleases used for human genome editing, and ex vivo genetically modified human cells.

### Definitions

The disclosure is described with respect to embodiments and with reference to certain drawings but the invention is not limited thereto, but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun, e.g., “a,” “an,” or “the,” this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The following terms or definitions are provided solely to aid in the understanding of the invention. Unless specifically defined herein, all terms used herein have the same meaning as they would to one skilled in the art hereof. Practitioners are directed to Sambrook et al., Molecular Cloning: A Laboratory Manual, 2nd ed., Cold Spring Harbor Press, Plainsview, N.Y. (1989); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

A “regulatory element” or “transcriptional regulatory element (TRE)” are used herein interchangeably and refer to transcriptional control elements, such as non-coding cis-acting transcriptional control elements, capable of regulating and/or controlling transcription of a gene, including cell type-specific transcription of a gene. Regulatory elements comprise at least one transcription factor binding site (TFBS), such as at least one binding site for a tissue-specific transcription factor and/or at least one binding site for a cardiac fibroblast-specific transcription factor. Typically, regulatory elements as used herein increase or enhance promoter-driven gene expression when compared to the transcription of the gene from the promoter alone, without the regulatory elements. Thus, regulatory elements and TREs comprise enhancer sequences, although it is to be understood that the regulatory elements enhancing transcription are not limited to typical far upstream enhancer sequences, but may occur at any distance of the gene they regulate. Indeed, it is known in the art that sequences regulating transcription may be situated either upstream (e.g., in the promoter region) or downstream (e.g., in the 3′UTR) of the gene they regulate in vivo, and may be located in the immediate vicinity of the gene or further away. Of note, although regulatory elements as disclosed herein typically are naturally occurring sequences, combinations of (parts of) such regulatory elements or several copies of a regulatory element, i.e., non-naturally occurring sequences, are themselves also envisaged as regulatory element. Regulatory elements as used herein may be part of a larger sequence involved in transcriptional control, e.g., part of a promoter sequence. However, regulatory elements alone are typically not sufficient to initiate transcription, but require a promoter to this end.

“Cardiac cell-specific” or “cardiac fibroblast-specific,” as used in the application, refers to the preferential or predominant ability of an enhancers ability to affect expression of an operably linked gene, nucleotide sequence encoding a gene product, (trans)gene or exogenous gene product (as RNA and/or polypeptide) in a cardiac cell and/or cardiac fibroblast as compared to other cell types, such as, for example, a hepatocyte and/or a cardiomyocyte. According to some embodiments, at least 50% of the exogenous gene product expression occurs within the cardiac fibroblast. According to some embodiments, gene product is expressed at a level and/or rate of at least two, at least three, at least four, at least five, at least six, at least seven, at least eight, at least nine, or at least ten times greater in cardiac fibroblasts than other cell types. In one embodiment, the other cell type is a cardiomyocyte. According to some embodiments, cardiac fibroblast-specific expression entails that there is little to no “leakage” of expressed gene product in other cell types.

As used herein, the term “expression cassette” refers to nucleic acid molecules that include one or more transcriptional control elements (such as, but not limited to promoters, enhancers and/or regulatory elements, polyadenylation sequences, and introns) that direct (trans)gene or exogenous gene product expression in one or more desired cell types, tissues or organs.

The term “operably linked” as used herein refers to the arrangement of various nucleic acid molecule elements relative to each such that the elements are functionally connected and are able to interact with each other. Such elements may include, without limitation, a promoter, an enhancer and/or a regulatory element, a polyadenylation sequence, one or more introns and/or exons, and a coding sequence of a gene of interest to be expressed (i.e., the transgene). The nucleic acid sequence elements, when properly oriented or operably linked, act together to modulate the activity of one another, and ultimately may affect the level of expression of the transgene. By modulate is meant increasing, decreasing, or maintaining the level of activity of a particular element. The position of each element relative to other elements may be expressed in terms of the 5′ terminus and the 3′ terminus of each element, and the distance between any particular elements may be referenced by the number of intervening nucleotides, or base pairs, between the elements.

As used in the application, the term “enhancer” refers to nucleic acid sequences that regulate, either directly or indirectly, the transcription of corresponding nucleic acid coding sequences to which they are operably linked (e.g., a transgene). An enhancer may function alone to regulate transcription or may act in concert with one or more other regulatory sequences (e.g., promoters, minimal promoters, or silencers). In the context of the application, an enhancer is typically operably linked to a transgene. When an enhancer as described herein is operably linked to a promoter and/or a transgene, the enhancer can (1) confer a significant degree of cardiac fibroblast specific expression in the heart in vivo (and/or in cardiac fibroblast cell lines in vitro) of the transgene, and/or (2) can increase the level of expression of the transgene in the heart (and/or in cardiac fibroblasts cell lines in vitro). An “enhancer” as described herein may, in some embodiments, function as a promoter in the absence of a promoter sequence other than the enhancer itself.

A “minimal promoter” as used herein is part of a full-size promoter still capable of driving expression, but lacking at least part of the sequence that contributes to regulating (e.g., tissue-specific) expression. This definition covers both promoters from which (tissue-specific) regulatory elements have been deleted—that are capable of driving expression of a gene but have lost their ability to express that gene in a tissue-specific fashion and promoters from which (tissue-specific) regulatory elements have been deleted that are capable of driving (possibly decreased) expression of a gene but have not necessarily lost their ability to express that gene in a tissue-specific fashion. Minimal promoters have been extensively documented in the art, such as the TATA box minimal promoter (Baumann et al. Mol Biotechnol. 45:241-247 (2010)).

The term “transgene” as used herein refers to particular nucleic acid sequences encoding a polypeptide or a portion of a polypeptide to be expressed in a cell into which the nucleic acid sequence is inserted. The polypeptide or portion of a polypeptide encoded by a transgene and expressed in a cell is also referred to herein as a “gene product” or “exogenous gene product.” However, it is also possible that transgenes are expressed as RNA, typically to lower the amount of a particular polypeptide in a cell into which the nucleic acid sequence is inserted. These RNA molecules include but are not limited to molecules that exert their function through RNA interference (shRNA, RNAi), micro-RNA regulation (miR), catalytic RNA, antisense RNA, RNA aptamers, etc. How the nucleic acid sequence is introduced into a cell is not essential to the invention, it may for instance be through integration in the genome or as an episomal plasmid. Of note, expression of the transgene may be restricted to a subset of the cells into which the nucleic acid sequence is inserted. The term “transgene” is meant to include (1) a nucleic acid sequence that is not naturally found in the cell (i.e., a heterologous nucleic acid sequence); (2) a nucleic acid sequence that is a mutant form of a nucleic acid sequence naturally found in the cell into which it has been introduced; (3) a nucleic acid sequence that serves to add additional copies of the same (i.e., homologous) or a similar nucleic acid sequence naturally occurring in the cell into which it has been introduced; or (4) a silent naturally occurring or homologous nucleic acid sequence whose expression is induced in the cell into which it has been introduced. By “mutant form” is meant a nucleic acid sequence that contains one or more nucleotides that are different from the wild-type or naturally occurring sequence, i.e., the mutant nucleic acid sequence contains one or more nucleotide substitutions, deletions, and/or insertions. In some cases, the transgene may also include a sequence encoding a leader peptide or signal sequence such that the transgene product will be secreted from the cell.

The term “vector” as used in the application refers to nucleic acid molecules, usually double-stranded DNA, which may have inserted into it another nucleic acid molecule (the insert nucleic acid molecule) such as, but not limited to, a cDNA molecule. The vector is used to transport the insert nucleic acid molecule into a suitable host cell. A vector may contain the necessary elements that permit transcribing the insert nucleic acid molecule, and, optionally, translating the transcript into a polypeptide. The insert nucleic acid molecule may be derived from the host cell, or may be derived from a different cell or organism. Once in the host cell, the vector can replicate independently of, or coincidental with, the host chromosomal DNA, and several copies of the vector and its inserted nucleic acid molecule may be generated. The term “vector” may thus also be defined as a gene delivery vehicle that facilitates gene transfer into a target cell. This definition includes both non-viral and viral vectors. Non-viral vectors include but are not limited to cationic lipids, liposomes, nanoparticles, polyethylene glycol (PEG), polyethylenimine (PEI), etc. Viral vectors are derived from viruses and include but are not limited to retroviral, lentiviral, adeno-associated viral, adenoviral, herpesviral, hepatitis viral vectors or the like. Typically, but not necessarily, viral vectors are replication-deficient as they have lost the ability to propagate in a given cell since viral genes essential for replication have been eliminated from the viral vector. However, some viral vectors can also be adapted to replicate specifically in a given cell, such as, e.g., a cancer cell, and are typically used to trigger the (cancer) cell-specific (onco)lysis.

### Enhancers

Gene expression, including that of transgenes, depends on transcriptional control. Genes present in the genome are not continuously expressed in all cell types: only a subset of them are expressed in a given cell at a given time point. Differential expression of genes is associated with differences in the chromatin, where the active regions (transcription and regulation) are open and the inactive regions are condensed. Native transcriptional control is a complex process where various cis- and trans-elements interact. Cis elements, such as promoters and enhancers, bind to transcription factors (TFs) complementary to their sequence. Typically several enhancers bind to different TFs, which bind together amongst each other and with co-factors to form a complex that interacts with a single promoter. The complexes that form this way, bind RNA polymerase II which can initiate transcription at a transcriptional start site. The combination of these processes leads to control under which conditions each different cell will transcribe individual genes and transgenes. Enhancer sequences, for example, can regulate cell type-specific gene and transgene expression.

TREs responsible for transcriptional control have been compiled and annotated in publicly available databases (Gao et al. Nucleic Acids Research 48:E58-D64 (2020); The ENCODE Project Consortium. Nature. 489:57-74 (2012)). Identification of cardiac-specific transcriptional regulatory elements have also been described (Jonsson et al. JACC Basic Transl Sci. 7:590-602 (2016)). Naturally occurring TREs can be used to design synthetic, tissue- or cell type-specific enhancer sequences. However, identification and validation of novel, cell type-specific enhancer sequences remains a challenge in the art, requiring extensive effort in the design and experimental validation of enhancer sequences. Attempts to identify and design enhancer sequences can have been made using computational approaches (Barr et al. BMC Syst Biol. 11:116 (2017); Niu et al. Front. Genet. 10:1305 (2019)). Enhancer properties, detailed enhancer-related transcriptional mechanisms, and naturally occurring cell specific enhancers are known in the art (Heinz et al. Nat Rev Cell Biol. 16:144-154 (2015); Ko Mol Cells. 40:169-177 (2017)).

In one aspect, the disclosure provides enhancer sequences. In some embodiments, the enhancer sequences are specific to cardiac cells. In some embodiments, the enhancers are specific to cardiac fibroblast cells. By “specific” it is meant that the enhancers facilitate expression of an operably linked gene, transgene, or nucleotide sequence encoding a gene product, as described herein, in only a target cell type. For example, an enhancer sequence that is specific to cardiac cells will facilitate expression of an operably linked transgene in cardiac cells but not in other cell types, e.g. hepatocytes. For example, an enhancer sequence that is specific to cardiac fibroblasts will facilitate expression of an operably linked transgene in cardiac fibroblasts but not in other cell types, e.g. cardiomyocytes.

In some embodiments, the enhancer sequence is operably linked to a transgene encoding a gene product. In some embodiments, two or more enhancer sequences are operably linked to a transgene encoding a gene product. In some embodiments, the enhancer sequence is operably linked to a nucleotide sequence encoding a gene product. The expression of a transgene encoding a gene product is facilitated or controlled by the enhancer sequence when operably linked to the enhancer sequence. In some embodiments, a transgene operably linked to the enhancer sequence is expressed at a level and/or rate of at least two, at least three, at least four, at least five, at least six, at least seven, at least eight, at least nine, or at least ten times greater in cardiac fibroblasts than cardiomyocytes. In some embodiments, a transgene operably linked to two or more enhancer sequences is expressed at a level and/or rate of at least two, at least three, at least four, at least five, at least six, at least seven, at least eight, at least nine, or at least ten times greater in cardiac fibroblasts than cardiomyocytes.

The enhancer sequences of the disclosure can be a range of lengths. In some embodiments, the enhancers can be about 50 nucleotides (nt), about 55 nt, about 60 nt, about 65 nt, about 70 nt, about 80 nt, about 85 nt, about 90 nt, about 95 nt, about 100 nt, about 105 nt, about 110 nt, about 115 nt, about 120 nt, about 125 nt, about 130 nt, about 135 nt, about 140 nt, about 145 nt, about 150 nt, about 155 nt, about 160 nt, about 165 nt, about 170 nt, about 175 nt, about 180 nt, about 185 nt, about 190 nt, about 195 nt, about 200 nt, about 205 nt, about 210 nt, about 215 nt, about 220 nt, about 225 nt, about 230 nt, about 235 nt, about 240 nt, about 245 nt, about 250 nt, about 255 nt, about 260 nt, about 265 nt, about 270 nt, about 275 nt, about 280 nt, about 285 nt, about 290 nt, about 295 nt, about 300 nt, about 305 nt, about 310 nt, about 315 nt, about 320 nt, about 325 nt, about 330 nt, about 335 nt, about 340 nt, about 345 nt, about 350 nt, about 355 nt, about 360 nt, about 365 nt, about 370 nt, about 375 nt, about 380 nt, about 385 nt, about 390 nt, about 395 nt, about 400 nt, about 405 nt, about 415 nt, about 420 nt, about 425 nt, about 430 nt, about 435 nt, about 440 nt, about 445 nt, about 450 nt, about 455 nt, about 460 nt, about 465 nt, about 470 nt, about 475 nt, about 480 nt, about 485 nt, about 490 nt, about 495 nt, about 500 nt, about 505 nt, about 515 nt, about 520 nt, about 525 nt, about 530 nt, about 535 nt, about 540 nt, about 545 nt, or about 550 nt. In reference to the length of an enhancer sequence, about is defined as #5 nt.

In some embodiments, the enhancer sequence shares at least 80%, at least 85%, at least 90%, at least 95%, at least 96%, at least 97%, at least 98%, at least 99%, or 100% identity to SEQ ID NOs: 1-21. In some embodiments, the enhancer sequences shares at least 80%, at least 85%, at least 90%, at least 95%, at least 98%, or 100% identity to SEQ ID NOs: 19. In some embodiments, the enhancer sequences shares at least 80%, at least 85%, at least 90%, at least 95%, at least 98%, or 100% identity to SEQ ID NOs: 20. In some embodiments, the enhancer sequences shares at least 80%, at least 85%, at least 90%, at least 95%, at least 98%, or 100% identity to SEQ ID NOs: 21. Illustrative enhancer sequences can be found in Table 1.

TABLE 1
Illustrative Enhancer Sequences

| SEQ ID | Size | |
|---|---|---|
| NO | (base) | Enhancer Sequence |
| 1 | 73 | CTCAGAGCGGGTGGGACCTGGGGAACCAGGAGATTCCGGCCTCTGCAACCGT |
| | | GGGGCATGCGGTGGAGGGGTG |
| 2 | 73 | CAGATGAGCCCTGGCAGCAGGTGGACTTGGGCACGTATTCGCTGTGGCAAAT |
| | | CATGTAATCTCTGGGGCTCAC |
| 3 | 150 | AAACACTGCTGCTTTTCTGCTTGCTTGGGCACTACATTGACAACAACCCCCT |
| | | GGCATATTTTTTTATTCTCCCTAACTTACTCCACCTCCCCATCCTCCTATCC |
| | | CCATATACATACTGCTCTGAGGAAATGGAAAATAGCTCCTTATTAA |
| 4 | 73 | GACGCCATGGGTGAGGGTCAGAGGCTTTTGGTGGGAGGTGGTGGTGCCTTCT |
| | | GCCTGGGAAGCCCCCCAGCAC |
| 5 | 88 | CCCAGTCCACCGTGCCCTGGACGCTGCTCATCCACTAGGACTGGGCTTCCGT |
| | | GTGCAGGGCTTCACCCTCGCTCTCCTCTCTGCCTGC |
| 6 | 280 | AAAAAAAAACAAAAAAAAAAACCCTATTATGTTTGTTTTCTATGCTTTTATG |
| | | ATGTTTTTGCATACATTTCTTTTAGATGTGTGTATCTTTTTTCTGTACACAC |
| | | CAGAGGAAGAAATAATAACAATGTCACATATGTTTGAGTCACTCTGAAATCA |
| | | TCAACTCTTGGTTCTTTGGTTCTAGCATGTCTTACACCTGTTTACTCTTACT |
| | | GTTTTAATGTCTGTTTACTCATTACAGATGATGTCAAGGAAGGGGATGGCTT |
| | | TCTCTTTTTTTTTTTTTCCTG |
| 7 | 73 | GCCCTGGCAGGGGAGTGGCCTGGAGGGGACACTCTCTAAAGACTGCTTCTAA |
| | | AGACCACGTCTTCTCATGACT |
| 8 | 73 | CCCATGCACCATGTTCCACACCGCACAACTACCTACACACCATGTTTCACAA |
| | | CACACAGCTACTCACATGCCA |
| 9 | 223 | GGGACATTCTGGGAATGTTTGGGGGATGGTGACTAGTTTAGTTTGGGAGAAG |
| | | CGTTGCCTGATCTTAGTGCAGCAGCAACTTGGGGTGTAGACCTGAAGCACCT |
| | | TGCCAACGGCACGGCACTGGGGAGCCACGGATGGCTCAGGAGCTCTGGGTGG |
| | | GGTCAGCAGAGCTAACTTGGGAAGGGAACTCGGGGCAGGGGTGGGAGGCAGC |
| | | ACAGAGACCTGAGGGC |
| 10 | 73 | GACCATTGGTTCCACTGTGCCGGACGGAACGTACTCACACCGGTGTCACCTC |
| | | AGAGCAGGTGTCCAGCTGAGC |
| 11 | 73 | AAATTTGATTAAAATATTATATTACCAAGGAGGAACAGAACACTATGTAGTT |
| | | ATGGGAAAAGTGGGGTTCTAC |
| 12 | 479 | TTCTCAGATCAATAACAATAACAAAAATCCTGGACTAGAAGAGAGAATCAAG |
| | | ATATTGCTTGTAATTTCACAAATGGCCGGAATTTCTGCAGTCCCCGGAGTAG |
| | | CAGGGCTCTAGTAAAAGGCTGCTTTCTCAGCCGCAAAATATTTCACAAACAT |
| | | GAGTTACTGATGGAGTCTCTACATTTGCCACTGAGCCAAGCTGGTTAGCAAA |
| | | GCTAGAACTTGGAATGTCCACCCAGAGAGATGACAAGTTGAGCCCCCAAGGG |
| | | AATGAGTTGACAGAGTTGAGTGGCGGCCACGTTCAGGGACCCGGTCTTAGAG |
| | | GCAGCGTGCCATGAGAGGAAGGTTCCTGAATGGGAGTCCACATCTTGGGTGC |
| | | CCCAGCCACTCGGCACCAGCTCACCCCACCCCCTGAGGCTTCAGCACCACAC |
| | | CATCCTCAGCCCCACACCGTCCTTCTCTGGGATGTCTATGTGGCCAGCTCAG |
| | | AACTCTACGTAT |
| 13 | 507 | TTCTTCTCTCTCTAAGATAAAGCCTGCTGGTTATAACAGAGTTGTTATTTTT |
| | | GGAGTCTTATTAAAGACTCTAGCACAGTCTAGCCAAAACAGAATGCTTTTCT |
| | | GGTGTACGGACAGAGCCTTTCTTTAACTCCACCCTGTAGTAGTTGGGTGCCT |
| | | ATGGCTGCAATGCCTTGGCCAGGGCATTGTGTGCTTCCAGAAGGTTCTCCTC |
| | | CCTTGATCACTGGTGGGGTTTTGATTTCCTATGAACTCAATTCCCTGACTAC |
| | | CCATAAAGAGATAACAAAAAGTACAAAAGATCCCAGTGAGGAGAAAGGACAA |
| | | CATACTTCCTCCAACACAGAGCATCCCTTTTTTAACTGAGTACTCATGAACA |
| | | TAGTTTCTACATGCTGTGGATCCTGACTCCTGTAATCTCCCCATCTCCCCGA |
| | | GGGCTCCTTCTTCTCATTCCCTCTGAGCCTATTCCTCACCCATCCTGACCTG |
| | | TATCATAGTCTGTGAACTCCTTGCCCCTCCCACCTGCTCC |
| 14 | 205 | CATCTTCTGTGAGGCTGCTGCACAGATAGGGTCTGTCCTCCTCACTGCCACA |
| | | AAGCCAAGCAACTCCATGTCACATGGGTCACGGCCTCAGGTGAAGGAGGCAC |
| | | TGTGTGAGGCAATGGTGGAATGTGAGGGAGTTGTGAGTGTGACGAGGCCTCA |
| | | GCGCCTGGCCCTCCTGAACAGGGATGCTCTGCTGCCCGCCCAGCCTGAGG |
| 15 | 206 | CACCCGGCCCTGACTCTGCAGCTAAAGACCCACATGCCTAGACCAGGGCGTC |
| | | TCCCTAGAAGGGCTTCTCCTGGAGATCTCTAGGGACTGACTCCTTGGCAGGC |
| | | TGGGCCGGGCTCTGGCTTCCATCCTTATCATTGGAAATCATCACCGGGCACT |
| | | GGAACTAGAGGGTAAGAGGGGAGGGAGAAGTGCAGAGGCTGGACAGGCAAA |
| 16 | 82 | ATGAATGGTGTTTATGTGCGTCTGCATGTGCGTGCCCATGTATGGTGTTTGT |
| | | GTGCACATGGGCATGCATATGTGAGGGTGT |
| 17 | 146 | CTCAGAGCGGGTGGGACCTGGGGAACCAGGAGATTCCGGCCTCTGCAACCGT |
| | | GGGGCATGCGGTGGAGGGGTGGCCCTGGCAGGGGAGTGGCCTGGAGGGGACA |
| | | CTCTCTAAAGACTGCTTCTAAAGACCACGTCTTCTCATGACT |
| 18 | 238 | AAACACTGCTGCTTTTCTGCTTGCTTGGGCACTACATTGACAACAACCCCCT |
| | | GGCATATTTTTTTATTCTCCCTAACTTACTCCACCTCCCCATCCTCCTATCC |
| | | CCATATACATACTGCTCTGAGGAAATGGAAAATAGCTCCTTATTAACCCAGT |
| | | CCACCGTGCCCTGGACGCTGCTCATCCACTAGGACTGGGCTTCCGTGTGCAG |
| | | GGCTTCACCCTCGCTCTCCTCTCTGCCTGC |
| 19 | 223 | AAACACTGCTGCTTTTCTGCTTGCTTGGGCACTACATTGACAACAACCCCCT |
| | | GGCATATTTTTTTATTCTCCCTAACTTACTCCACCTCCCCATCCTCCTATCC |
| | | CCATATACATACTGCTCTGAGGAAATGGAAAATAGCTCCTTATTAAGCCCTG |
| | | GCAGGGGAGTGGCCTGGAGGGGACACTCTCTAAAGACTGCTTCTAAAGACCA |
| | | CGTCTTCTCATGACT |
| 20 | 161 | GACGCCATGGGTGAGGGTCAGAGGCTTTTGGTGGGAGGTGGTGGTGCCTTCT |
| | | GCCTGGGAAGCCCCCCAGCACCCCAGTCCACCGTGCCCTGGACGCTGCTCAT |
| | | CCACTAGGACTGGGCTTCCGTGTGCAGGGCTTCACCCTCGCTCTCCTCTCTG |
| | | CCTGC |
| 21 | 161 | CTCAGAGCGGGTGGGACCTGGGGAACCAGGAGATTCCGGCCTCTGCAACCGT |
| | | GGGGCATGCGGTGGAGGGGTGCCCAGTCCACCGTGCCCTGGACGCTGCTCAT |
| | | CCACTAGGACTGGGCTTCCGTGTGCAGGGCTTCACCCTCGCTCTCCTCTCTG |
| | | CCTGC |

Enhancers of the invention are not limited to specific sequences referred to in the specification but also encompass their structural and functional analogs/homologues. Such analogs may contain truncations, deletions, insertions, as well as substitutions of one or more nucleotides introduced either by directed or by random mutagenesis. Truncations may be introduced to delete one or more binding sites for known transcriptional repressors. Additionally, such sequences may be derived from sequences naturally found in nature that exhibit a high degree of identity to the sequences in the invention. A nucleic acid of 20 nt or more will be considered to have high degree of identity to a promoter/enhancer sequence of the invention if it hybridizes to such promoter/enhancer sequence under stringent conditions. Alternatively, a nucleic acid will be considered to have a high degree of identity to a promoter/enhancer sequence of the invention if it comprises a contiguous sequence of at least 20 nt, which has percent identity of at least 60%, 65%, 70%, 75%, 80%, 85%, 90%, 95%, or more as determined by standard alignment algorithms such as, for example, Basic Local Alignment Tool (BLAST) described in Altshul et al., J. Mol. Biol. 215:403-410 (1990), the algorithm of Needleman et al., J. Mol. Biol. 48:444-453 (1970), or the algorithm of Meyers et al., Comput. Appl. Biosci. 4:11-17 (1988). Non-limiting examples of analogs, e.g., homologous promoters sequences and homologous enhancer sequences derived from various species, are described in the present specification.

### Polynucleotides

The disclosure provides polynucleotides comprising enhancer sequences described herein. A recombinant polynucleotide can be an isolated polynucleotide, a synthetic polynucleotide, and/or a polynucleotide amplified by expression in a host cell for isolation. An isolated nucleotide can be, for example, substantially or completely free of contaminants. The polynucleotides described herein can be manufactured, produced, propagated, amplified, synthesized, characterized, identified and/or isolated by any technique known in the art. For example, the polynucleotides provided herein can be propagated and isolated from a host cell. A “host cell” refers to a living cell into which a heterologous polynucleotide sequence, e.g. the enhancer sequences described herein, is to be or has been introduced. The living cell can include a cultured cell and/or a cell within a living organism. Means for introducing the heterologous polynucleotide sequence into the cell are well known, e.g., transfection, electroporation, calcium phosphate precipitation, microinjection, transformation, viral infection, and/or the like. The heterologous polynucleotide sequence to be introduced into the cell can be a replicable expression vector or cloning vector. In some embodiments, host cells can be engineered to incorporate a desired gene on its chromosome or in its genome. Many host cells can be employed (e.g., CHO cells) and serve as hosts as is well known in the art. Recombinant techniques and methods are described, for example, in Sambrook et al. Molecular Cloning: A Laboratory Manual; Cold Spring Harbor Laboratory Press, 4th edition 2012.

In some embodiments, the polynucleotide comprises a cardiac cell-specific enhancer sequence. In some embodiments, the polynucleotide comprises a cardiac fibroblast-specific enhancer sequence. In some embodiments, the polynucleotide comprises two or more cardiac cell-specific enhancer sequences. In some embodiments, the polynucleotide comprises two or more cardiac fibroblast-specific enhancer sequences. In some embodiments, polynucleotide comprises an enhancer sequence shares at least 80%, at least 85%, at least 90%, at least 95%, at least 96%, at least 97%, at least 98%, at least 99%, or 100% identity to SEQ ID NOs: 1-21. In some embodiments, the polynucleotide comprises an enhancer sequence that shares at least 80%, at least 85%, at least 90%, at least 95%, at least 98%, or 100% identity to SEQ ID NOs: 19. In some embodiments, the polynucleotide comprises an enhancer sequence that shares at least 80%, at least 85%, at least 90%, at least 95%, at least 98%, or 100% identity to SEQ ID NOs: 20. In some embodiments, the polynucleotide shares at least 80%, at least 85%, at least 90%, at least 95%, at least 98%, or 100% identity to SEQ ID NOs: 21.

In some embodiments, the polynucleotide comprises a transgene encoding a gene product operably linked to a enhancer sequence. In some embodiments, the polynucleotide comprises a transgene encoding a gene product operably linked to at least two enhancer sequences. The enhancer can be, for example, the cardiac-cell specific enhancers described herein. The enhancer can be, for example, the cardiac fibroblast-specific enhancers described herein. The polynucleotide comprising a sequence encoding a gene product operably linked to the enhancer can optionally include sequence elements that confer additional properties or capabilities related to the manipulation or use of the polynucleotide such as, for example, recombinant manipulation, propagation and/or amplification of the polynucleotide, expression of the gene product in a host cell, and/or detection of the polynucleotide or gene product. In some embodiments the polynucleotide comprises restriction sites for targeted cleavage by an endonuclease. The polynucleotide can include any restriction site. Restriction sites are well known in the art and can be, for example, Xbal, SbfI, Agel, and/or EcoRI restriction sites. In some embodiments, the polynucleotide comprises a reporter gene. In some embodiments, the reporter gene is green fluorescent protein (GFP). In some embodiments, the polynucleotide comprises a response element. Response elements can be sequences of DNA recognized by the DNA binding domain transcription factors, that facilitate gene expression by, for example, initiation or elongation of an RNA transcript. In some embodiments, the response elements are operably linked to the enhancer sequences described herein. In some embodiments, the response elements are operably linked to the sequence encoding a gene product or transgene. In some embodiments, the response elements are operably linked to the enhancer sequences described herein and the sequence encoding a gene product or transgene. In some embodiments, the polynucleotides described herein are inserted into a vector. Sequence elements that confer additional properties or capabilities related to the manipulation or use of the polynucleotide described herein can be found in Sambrook et al. Molecular Cloning: A Laboratory Manual; Cold Spring Harbor Laboratory Press, 4th edition 2012.

In some embodiments, the polynucleotide comprises an enhancer sequence operably linked to a transgene encoding a gene product. In some embodiments, the polynucleotide comprises two or more enhancer sequences operably linked to a transgene encoding a gene product. In some embodiments, the polynucleotide comprises an enhancer sequence operably linked to a transgene encoding a gene product. The expression of a transgene encoding a gene product is facilitated or controlled by the enhancer sequence when operably linked to the enhancer sequence. In some embodiments, the polynucleotide comprises a transgene operably linked to the enhancer sequence, wherein the transgene is expressed at a level and/or rate of at least two, at least three, at least four, at least five, at least six, at least seven, at least eight, at least nine, or at least ten times greater in cardiac fibroblasts than cardiomyocytes. In some embodiments, the polynucleotide comprises a transgene operably linked to two or more enhancer sequences, wherein the transgene is expressed at a level and/or rate of at least two, at least three, at least four, at least five, at least six, at least seven, at least eight, at least nine, or at least ten times greater in cardiac fibroblasts than cardiomyocytes.

In some embodiments, the polynucleotide further comprises transcriptional regulatory elements (TREs). In some embodiments, the polynucleotide comprises a TRE operably linked to the enhancer sequences described herein. In some embodiments, the polynucleotide comprises a TRE operably linked to at least two enhancer sequences described herein. In some embodiments, the polynucleotide comprises a TRE operably linked to a transgene encoding a gene product. In some embodiments, the polynucleotide comprises a TRE operably linked to an enhancer described herein and a transgene encoding a gene product. In some embodiments, the polynucleotide comprises a TRE operably linked to at least two enhancer sequences described herein and a transgene encoding a gene product. In some embodiments, the TRE polynucleotide comprises a promoter. In some embodiments, the TRE is a promoter. In some embodiments, the polynucleotide comprises a minimal promoter. In some embodiments, the polynucleotide comprises a super core promoter.

### Transgenes

In one aspect, the enhancers described herein are operably linked to a transgene encoding a product. A transgene can be a gene or nucleotide sequence that encodes a product, or functional fragment thereof. A product can be, for example, a polypeptide or a non-coding nucleotide. By non-coding nucleotide, it is meant that the sequence transcribed from the gene or nucleotide sequence is not translated into a polypeptide. In some embodiments, the product encoded by the gene or nucleotide operably linked to an enhancer described herein is a non-coding polynucleotide. A non-coding polynucleotide can be an RNA, such as for example a microRNA (miRNA or mIR), short hairpin RNA (shRNA), long non-coding RNA (InRNA), short interfering RNA (siRNA). In some embodiments, the enhancers of the disclosure are operably linked to a transgene that encodes a product natively expressed by a cardiac fibroblast. In some embodiments, the enhancers of the disclosure are operably linked to a gene or nucleotide sequence expressed in a cell type other than a cardiac fibroblast. Without limitation, cell types other than cardiac fibroblasts can be from any multicellular organism, single-celled organism, or microorganism.

In some embodiments, the enhancers of the disclosure are operably linked to one or more transgenes encoding a product. Without limitation, examples of transgenes and/or products encoded by transgenes can be cadherins, connexins, Cx43, growth factors such as fibroblast growth factor (FGF)-2 and transforming growth factor-β, cytokines such as interleukin (IL)-1β and the IL-6 family, leukemia inhibitory factor, cardiotrophin-1, cardiogenic transcription factors, insulin-like growth factor, GATA4, MEF2C, TBX5, ESRRG, MESP1, MYOCD, ZFPM2, HAND2, miR-1, miR-133, Oct4, Sox2, Klf4, c-Myc, SRF, SMARCD3, Nkx2-5, Akt, PKB, Baf60c, BMP4, miR-208, miR-499. It is appreciated that the transgenes described herein are non-limiting and useful transgenes may be discovered. In some embodiments, the transgene encodes a polypeptide. In some embodiments, the transgene encodes a non-coding polynucleotide such as, for example, a microRNA (miRNA or mIR).

In some embodiments, the enhancers of the disclosure are operably linked to one or more transgenes encoding a product. The transgene may comprise a DNA sequence encoding a polypeptide or non-coding product involved in reprogramming or differentiating cells, metabolic diseases, disorders and diseases of cardiopulmonary system, heart arrhythmia, cardiomyopathy, congenital heart defects, coronary artery disease, heart infections, and/or atherosclerosis. Vectors of the invention may include a transgene containing a sequence coding for a therapeutic polypeptide. For gene therapy, such a transgene is selected based upon a desired therapeutic outcome. It may encode, for example, antibodies, hormones, enzymes, receptors, or other proteins of interest or their fragments.

In some embodiments, the enhancers of the disclosure are operably linked to one or more transgenes encoding a product. Transgenes can encode products that modulate the phenotype or functional effects of cardiac cells, e.g. cardiac fibroblasts and cardiomyocytes. Without limitation, transgenes can encode products that can, for example, mediate autocrine or paracrine signaling between cardiac fibroblasts and cardiomyocytes, effect the extracellular matrix (ECM) in cardiac tissue, modulate the cardiogenic transcriptional network, induced collagen production, modulate proliferation, affect hypertrophy, reduce fibrosis, regenerate myocardial tissue, and/or reprogram cardiac fibroblasts into cardiomyocytes.

### Vectors

The disclosure provides vectors comprising an enhancer sequence described herein. In general, there are no known limitations on the use of the enhancer sequences of the invention in any vector. In some embodiments, the enhancer sequences described herein are incorporated in non-viral plasmid-based vectors. In some embodiments, the enhancer sequences described herein are incorporated into a viral vector such as derived from adenoviruses, adeno-associated viruses (AAV), or retroviruses, including lentivirus such as the human immunodeficiency (HIV) virus. In some embodiments, the enhancer sequences described herein are incorporated into a cloning vector.

Reference to a vector or other DNA sequences as “recombinant” merely acknowledges the operable linkage of DNA sequences which are not typically operably linked as isolated from or found in nature. Regulatory (expression and/or control) sequences are operatively linked to a nucleic acid coding sequence when the expression and/or control sequences regulate the transcription and, as appropriate, translation of the nucleotide sequence. Thus expression and/or control sequences can include promoters, enhancers, transcription terminators, a start codon (i.e., ATG) 5′ to the coding sequence, splicing signals for introns and stop codons.

In some embodiments, the vector comprises a polynucleotide comprising an enhancer sequence described herein and an operably linked transgene encoding a product. The vector may be used for transfecting or transducing a host cell, wherein the transfection or transduction transmits the polynucleotide comprising an enhancer sequence. The sequence elements arranged in a definite pattern of organization such that the expression of genes/gene products that are operably linked to these elements can be predictably controlled. Typically, they are transmissible polynucleotide sequences (e.g., plasmid or virus) into which a segment of foreign DNA, e.g. enhancers described herein and/or a transgene, can be spliced in order to introduce the foreign DNA into host cells to promote its replication and/or transcription.

A cloning vector is a DNA sequence (typically a plasmid or phage) which is able to replicate autonomously in a host cell, and which is characterized by one or a small number of restriction endonuclease recognition sites. A foreign DNA fragment, e.g. enhancers described herein and/or a transgene, may be spliced into the vector at these sites in order to bring about the replication and cloning of the fragment. The vector may contain one or more markers suitable for use in the identification of transformed cells. For example, markers may provide tetracycline or ampicillin resistance.

An expression vector is similar to a cloning vector but is capable of inducing the expression of the DNA that has been cloned into it, after transformation into a host. The cloned DNA is usually placed under the control of (i.e., operably linked to) certain regulatory sequences such as promoters or enhancers. Promoter sequences may be constitutive, inducible or repressible.

The disclosure provides the use of any viral vector, e.g. an adeno-associated viral vector (AAV). The AAV viral vector may be any serotype for introduction of constructs comprising the enhancer sequences described herein. A large number of AAV vectors are known in the art. In generating recombinant AAV viral vectors, non-essential genes are replaced with a gene encoding a protein or polypeptide of interest. AAV serotypes include AAV2 and others that each have specific tropisms and transduction capabilities (Davidson et al (2000), PNAS 97(7)3428-32; Passini et al (2003), J. Virol 77(12):7034-40). In one aspect, the present invention is directed to AAV vectors and methods that allow optimal AAV vector-mediated delivery of the enhancers or polynucleotides described herein in vitro or in vivo.

The vector typically comprises an origin of replication and the vector may or may not in addition comprise a “marker” or “selectable marker” function by which the vector can be identified and selected. While any selectable marker can be used, selectable markers for use in recombinant vectors are generally known in the art and the choice of the proper selectable marker will depend on the host cell. Examples of selectable marker genes which encode proteins that confer resistance to antibiotics or other toxins include, but are not limited to ampicillin, methotrexate, tetracycline, neomycin (Southern et al., J. Mol. Appl. Genet. 1:327-41 (1982)), mycophenolic acid (Mulligan et al., Science 209:1422-27 (1980)), puromycin, zeomycin, hygromycin (Sugden et al., Mol Cell Biol. 5(2):410-3 (1985)) and G418. As will be understood by those of skill in the art, expression vectors typically include an origin of replication, a promoter operably linked to the coding sequence or sequences to be expressed, as well as ribosome binding sites, RNA splice sites, a polyadenylation site, and transcriptional terminator sequences, as appropriate to the coding sequence(s) being expressed.

Adeno-associated virus (AAV) is a helper-dependent human parvovirus which is able to infect cells latently by chromosomal integration. Because of its ability to integrate chromosomally and its nonpathogenic nature, AAV has significant potential as a human gene therapy vector. For use in practice, rAAV virions may be produced using standard methodology, known to those of skill in the art and are constructed such that they include, as operatively linked components in the direction of transcription, enhancer sequences described herein, control sequences including transcription initiation and termination sequences, e.g. promoters, and transgenes of interest. More specifically, the recombinant AAV vectors of the disclosure comprise: a packaging site enabling the vector to be incorporated into replication-defective AAV virions, wherein the vector comprises an expression cassette comprising the enhancer sequences described herein and, optionally, a transgene and/or other sequence elements described herein. AAV vectors are constructed such that they may also include, as operatively linked components in the direction of transcription, control sequences including transcription initiation and termination sequences, e.g. TREs such as promoters. These components are flanked on the 5′ and 3′ end by functional AAV ITR sequences. By “functional AAV ITR sequences” is meant that the ITR sequences function as intended for the replication and packaging of the AAV virion.

Recombinant AAV vectors are also characterized in that they are capable of directing the expression and production of transgenes operably linked to the enhancers described herein. Thus, the recombinant vectors may comprise sequences of AAV for encapsidation and the physical structures for infection of the recombinant AAV (rAAV) virions. Hence, AAV ITRs for use in the vectors of the invention need not have a wild-type nucleotide sequence (e.g., as described in Kotin, Hum. Gene Ther., 5:793-801, 1994), and may be altered by the insertion, deletion or substitution of nucleotides or the AAV ITRs may be derived from any of several AAV serotypes. Generally, an AAV vector is a vector derived from an adeno-associated virus serotype, including without limitation, AAV-1, AAV-2, AAV-3, AAV-4, AAV-5, AAV-6, AAV-7, AAV-8, etc. The rAAV vectors may have the wild type REP and CAP genes deleted, in whole or part, but retain functional flanking ITR sequences.

Typically, an AAV expression vector is introduced into a producer cell, followed by introduction of an AAV helper construct, where the helper construct includes AAV coding regions capable of being expressed in the producer cell and which complement AAV helper functions absent in the AAV vector. The helper construct may be designed to down regulate the expression of the large Rep proteins (Rep78 and Rep68), typically by mutating the start codon following p5 from ATG to ACG, as described in U.S. Pat. No. 6,548,286, expressly incorporated by reference herein. This is followed by introduction of helper virus and/or additional vectors into the producer cell, wherein the helper virus and/or additional vectors provide accessory functions capable of supporting efficient rAAV virus production. The producer cells are then cultured to produce rAAV. These steps are carried out using standard methodology. Replication-defective AAV virions encapsulating the recombinant AAV vectors of the instant invention are made by standard techniques known in the art using AAV packaging cells and packaging technology. Examples of these methods may be found, for example, in U.S. Pat. Nos. 5,436,146; 5,753,500, 6,040,183, 6,093,570 and 6,548,286, expressly incorporated by reference herein in their entirety. Further compositions and methods for packaging are described in Wang et al. (US 2002/0168342), also incorporated by reference herein in its entirety and include those techniques within the knowledge of those of skill in the art.

Approximately 40 serotypes of AAV are currently known, however, new serotypes and variants of existing serotypes are still being identified today and are considered within the scope of the present invention. See Gao et al (2002), PNAS 99(18): 11854-6; Gao et al (2003), PNAS 100(10):6081-6; Bossis and Chiorini (2003), J. Virol. 77(12):6799-810). Different AAV serotypes are used to optimize transduction of particular target cells or to target specific cell types within a particular target tissue. The use of different AAV serotypes may facilitate targeting of diseased tissue, e.g. cardiac tissue. Particular AAV serotypes may more efficiently target and/or replicate in specific target tissue types or cells. A single self-complementary AAV vector can be used in practicing the invention in order to increase transduction efficiency and result in faster onset of transgene expression (McCarty et al., Gene Ther. 2001 August; 8(16):1248-54).

Viral vector constructs comprising polynucleotides and enhancer sequences described herein, e.g. lentiviral and AAV vectors, may be introduced into cells in vitro, in vivo, or ex vivo using standard methodology known in the art. Such techniques include transfection using calcium phosphate, microinjection into cultured cells (Capecchi, Cell 22:479-488 (1980)), electroporation (Shigekawa et al., BioTechn., 6:742-751 (1988)), liposome-mediated gene transfer (Mannino et al., BioTechn., 6:682-690 (1988)), lipid-mediated transduction (Felgner et al., Proc. Natl. Acad. Sci. USA 84:7413-7417 (1987)), and nucleic acid delivery using high-velocity microprojectiles (Klein et al., Nature 327:70-73 (1987)). AAV vectors may be administered in vivo by injection, e.g. intravenous injection, as part of a composition comprising a pharmaceutically acceptable carriers, diluents, and excipients.

Also disclosed herein are methods of transfecting cardiac tissue where such methods utilize the vectors of the invention. It will be understood that vectors of the invention are not limited by the type of the transfection agent in which to be administered to a subject or by the method of administration. Without limitation, transfection agents may contain compounds that reduce the electrostatic charge of the cell surface and the polynucleotide itself, or increase the permeability of the cell wall. Examples include cationic liposomes, calcium phosphate, polylysine, vascular endothelial growth factor (VEGF), etc. Hypertonic solutions containing, for example, NaCl, sugars, or polyols, can also be used to increase the extracellular osmotic pressure thereby increasing transfection efficiency. Transfection agent may also include enzymes such as proteases and lipases, mild detergents and other compounds that increase permeability of cell membranes. The methods disclosed herein are not limited to any particular composition of the transfection agent and can be practiced with any suitable agent so long as it is not toxic to the subject or its toxicity is within acceptable limits. Non-limiting examples of suitable transfection agents are given in this specification.

### Cells

The disclosure provides an isolated cell comprising the enhancers, polynucleotides, and vectors described herein. In some embodiments, the cell comprises a polynucleotide comprising an enhancer described herein. In some embodiments, the cell comprises a vector comprising an enhancer described herein. In some embodiments, the cell is a cardiac cell. In some embodiments, the cell is a cardiac fibroblast. In some embodiments, the cell is a cardiomyocyte. In some embodiments, the cell is an induced pluripotent stem cell. In some embodiments, the cell is an induced pluripotent stem cell-derived cardiac cell. The cell disclosed herein can be transfected or transduced with polynucleotides and/or vectors comprising an enhancer or combination of enhancers described herein. Transfected or transduced cells may be used, for example, to determine the physiological function of a transgene; to treat a disease in a subject in need thereof; to determine biosynthesis and intracellular transport of proteins encoded by transgenes; to determine the role of a transgene in inter- and intracellular interactions among a plurality of cells in a culture, in a tissue, in an organ, or in an organism. The cell may be part of a composition for subsequent re-implantation into a subject, as part of a gene therapy.

Host cells that can be used with the polynucleotides and vectors comprising the enhancer sequences described herein can be any cell type from any organ system, e.g., from muscle, liver, kidney, bone marrow, skin, etc. Cells are found and can be isolated from any vertebrate species, including, without limitation, human, orangutan, monkey, chimpanzee, dog, cat, rat, rabbit, mouse, horse, cow, pig, elephant, etc. Examples of tissue from which such cells can be isolated include, for example, cardiac, placenta, umbilical cord, bone marrow, skin, muscle, periosteum, or perichondrium. Alternatively, the host cell can be a prokaryotic cell, e.g., a bacterial cell such as E. coli, that is used, for example, to propagate vectors comprising the enhances, polynucleotides, and vectors described herein.

It may be desirable in certain circumstances to utilize progenitor cells such as stem cells or induced pluripotent stem cells rather than fully differentiated cell types. Certain cardiac cells, e.g. cardiomyocytes, can be derived from such cells, for example, by inducing their differentiation in tissue culture. The present invention encompasses not only fully differentiated and progenitor cells, but also cells that can be trans-differentiated into cardiomyocytes, e.g., cardiac fibroblasts.

In some embodiments, the cell comprises an enhancer operably linked to a transgene. In some embodiments, the cell is a cardiac fibroblast, wherein the transgene is expressed at a level and/or rate of at least two, at least three, at least four, at least five, at least six, at least seven, at least eight, at least nine, or at least ten times greater than a cardiomyocyte, wherein the cardiac fibroblast and cardiomyocyte both comprise the enhancer operably linked to the transgene. In some embodiments, the cell is a cardiac fibroblast, wherein the transgene is expressed at a level and/or rate of at least two times greater in cardiac fibroblasts than a cardiomyocyte, wherein the cardiomyocyte comprises the enhancer operably linked to the transgene.

The heart comprises tissue, i.e. cardiac tissue, containing a heterogeneous population of cell types, including cardiomyocytes and cardiac fibroblasts (Jonsson et al. JACC Basic Transl Sci. 1:590-602 (2016)). Although cardiomyocytes (CMs) occupy most of the tissue volume and provide the mechanical force delivered by the heart, they are largely outnumbered by nonmyocyte cells (30% vs. 70%), part of which are cardiac fibroblasts (CFs). Cross-sectional confocal microscopy of ventricular tissue has shown that each CM is in the direct vicinity of at least one CF, reflecting a significant role for CFs in the heart. CF function includes providing a supportive environment for CMs, such as by regulation of the extracellular matrix (ECM). In some embodiments, the cardiac fibroblast-specific enhancers described herein are operably linked to a gene or nucleotide that encodes a product that regulates the extracellular matrix.

In some embodiments, cardiac fibroblast-specific enhancers of the disclosure are operably linked to transgene sequences that encode a product capable of mediating signaling between cardiac fibroblasts and cardiomyocytes. In some embodiments, cardiac fibroblast-specific enhancers of the disclosure are operably linked to a gene or nucleotide sequence whose expression product increases signaling between cardiac fibroblasts and cardiomyocytes. In some embodiments, cardiac fibroblast-specific enhancers of the disclosure are operably linked to a gene or nucleotide sequence whose expression product reduces or eliminates signaling between cardiac fibroblasts and cardiomyocytes. Without being bound by theory, CFs communicate with CMs through at least three different and non-limiting mechanisms. For example, direct cell-to-cell contact, in which the formation of adherens junctions (cadherins) and gap junctions (connexins) play a crucial role in signaling. In some embodiments, cardiac fibroblast-specific enhancers of the disclosure are operably linked to genes or nucleotide sequences that encode a product capable of mediating the formation of adherens junctions. A non-limiting example of a gene that encodes a gene product capable of mediating gap junctions is Cx43 (Kizana et al. Gene Therapy. 13:1611-1615 (2006)). In some embodiments, cardiac fibroblast-specific enhancers of the disclosure are operably linked to genes or nucleotide sequences that encode a product capable of mediating the formation of gap junctions. Another example is by paracrine or autocrine secretion of growth factors such as fibroblast growth factor (FGF)-2/basic FGF and transforming growth factor-β or important cytokines such as interleukin (IL)-1B and the IL-6 family, including leukemia inhibitory factor and cardiotrophin-1. In some embodiments, cardiac fibroblast-specific enhancers of the disclosure are operably linked to genes or nucleotide sequences that encode a product capable of mediating autocrine or paracrine signaling between cardiac fibroblasts and cardiomyocytes. In the third method, cells indirectly relay signals via the extracellular matrix (ECM) by modulating its composition and quantity by secretion or degradation of the ECM building blocks. In some embodiments, cardiac fibroblast-specific enhancers of the disclosure are operably linked to genes or nucleotide sequences that encode a product that modulates the composition or quantity of ECM building blocks.

In some embodiments, cardiac fibroblast-specific enhancers of the disclosure can be used to mediate the presence or absence of phenotypic features of cardiac fibroblasts. Common phenotypic features of fibroblasts are the lack of a basement membrane, profound granular material in the cytoplasm scattered along a large Golgi apparatus, and a substantial rough endoplasmic reticulum. Another feature of CFs is their ability to transform into an active state; the myofibroblast. Myofibroblasts express smooth muscle cell markers (e.g., smooth muscle actin [SMA]) and may contract Myofibroblasts have also been implicated in wound contraction, fibrosis, and scar healing and are a source of cytokines and growth factors, such as IL-6 and transforming growth factor-β. In some embodiments, cardiac fibroblast-specific enhancers of the disclosure are operably linked to a gene or nucleotide sequence whose expression product increases phenotypic features of cardiac fibroblasts. In some embodiments, cardiac fibroblast-specific enhancers of the disclosure are operably linked to a gene or nucleotide sequence whose expression product reduces or eliminates phenotypic features of cardiac fibroblasts.

In some embodiments, the cardiac fibroblasts-specific enhancers described herein are operably linked to a gene or nucleotide sequence encoding a product that modulates cardiac tissue phenotype. Cardiac fibroblasts express common core fibroblast genes, and uniquely have a specific gene expression profile involving the cardiogenic transcriptional network (Furtado et al. Circ Res. 114:1422-1434 (2014)). In some embodiments, the cardiac fibroblast-specific enhancers described herein modulate the expression of genes in the cardiogenic transcriptional network. In some embodiments, the cardiac-specific enhancers described herein are operably linked to a gene or nucleotide encoding a product that increases expression of genes in the cardiogenic transcriptional network. In some embodiments, the cardiac-specific enhancers described herein are operably linked to a gene or nucleotide encoding a product that reduces or eliminates expression of genes in the cardiogenic transcriptional network. Furthermore, regional differences exist in which CFs from the atrium and the ventricle express different cardiogenic transcription factors (TFs) (Burstein et al. Circulation. 117:1630-1641 (2008)). In some embodiments, the cardiac fibroblast-specific enhancers described herein are operably linked to cardiogenic transcription factors. Important differences have also been found between rat CFs from the embryonic heart compared with the adult heart, with a differential response in insulin-like growth factor-induced collagen production (Diaz-Araya et al. Cell Commun Adhes. 10:155-165 (2003)). In some embodiments, the cardiac fibroblast-specific enhancers described herein are operably linked to a gene or nucleotide sequence encoding a product that increases insulin-like growth factor-induced collagen production. In some embodiments, the cardiac fibroblast-specific enhancers described herein are operably linked to a gene or nucleotide sequence encoding a product that decreases or eliminates insulin-like growth factor-induced collagen production. The influence of CFs on co-cultured CMs also varies depending on age. Embryonic CFs increase proliferation of CMs, whereas adult CFs induce hypertrophy (Ieda et al. Dev Cell. 16:233-244 (2009)). In some embodiments, the cardiac fibroblast-specific enhancers described herein are operably linked to a gene or nucleotide sequence encoding a product that decreases or eliminates cardiac fibroblast mediated proliferation of cardiomyocytes. In some embodiments, the cardiac fibroblast-specific enhancers described herein are operably linked to a gene or nucleotide sequence encoding a product that increases cardiac fibroblast mediated proliferation of cardiomyocytes. In some embodiments, the cardiac fibroblast-specific enhancers described herein are operably linked to a gene or nucleotide sequence encoding a product that decreases or eliminates cardiac fibroblast mediated hypertrophy. In some embodiments, the cardiac fibroblast-specific enhancers described herein are operably linked to a gene or nucleotide sequence encoding a product that increases cardiac fibroblast mediated hypertrophy.

Cardiac fibroblasts can be reprogrammed into cardiomyocytes (Srivastava and Berry 2013; Fu and Srivastava 2015; Sahara et al. 2015; Srivastava and Yu 2015). Reprogramming cardiac fibroblasts to cardiomyocytes can potentially reduce fibrosis and to induce the regeneration of the myocardial tissue through the generation of novel cardiomyocytes following myocardial infarction. The direct reprogramming of fibroblasts/myofibroblasts into induced cardiomyocyte-like cells has been shown by several groups both in vitro and in vivo. Direct reprogramming has been accomplished using many sets of reprogramming factors (Talman et al. Cell Tissue Res. 365:563-581 (2016)). Sets of factors used to reprogram fibroblasts to cardiomyocytes in humans include: GATA4, MEF2C, TBX5, ESRRG, MESP1, MYOCD, ZFPM2; GATA4, HAND2, MYOCD, TBX5, miRNA-1, miRNA-133; GATA4, MEF2C, TBX5, MESP1, MYOCD; and GATA4, MEF2C, TBX5, MESP1, MYOCD. Sets of factors used to reprogram fibroblasts to cardiomyocytes in mice include: Gata4, Mef2c, Tbx5; Transient Oct4, Sox2, Klf4, c-Myc; Gata4, Mef2c, Tbx5; Tbx5, Mef2c, Myocd; Gata4, Hand2, Mef2c, Tbx5; Gata4, Mef2c, Tbx5; Hand2, Nkx2-5, Gata4, Mef2c, Tbx5; GATA4, TBX5, MEF2C, SRF, MYOCD, SMARCD3, Mesp1; Gata4, Hand2, Mef2c, Tbx5; Gata4, Hand2, M3-Mef2c, Tbx5; Hand2, Nkx2-5, Gata4, Mef2c, Tbx5; Gata4, Mef2c, Tbx5, miRNA-133; Gata4, Hand2, Mef2c, Tbx5; Transient Oct4, Sox2, Klf4, c-Myc; Oct4; Polycistronic Mef2c-Gata4-Tbx; Gata4, Hand2, Mef2c, Tbx5; Gata4, Hand2, Mef2c, Tbx5, Akt/PKB; Polycistronic Mef2c-Gata4-Tbx5+Bmil shRNA; Gata4, Mef2c, Tbx5; Gata4, Hand2, Mef2c, Tbx5; Polycistronic Mef2c-Gata4-Tbx5; Mesp1, Tbx5, Gata4, Nkx2-5, Baf60c, leukemia inhibitory factor (LIF); Transient Oct4, Sox2, Klf4, c-Myc; BMP4; miRNA-1, miRNA-133, miRNA-208, miRNA-499. Sets of factors used to reprogram fibroblasts to cardiomyocytes in dogs include: Transient Oct4, Sox2, Klf4, c-Myc. Sets of factors used to reprogram fibroblasts to cardiomyocytes in rat include: Triplet Gata4-Mef2c-Tbx5, VEGF. It is appreciated in the art that the sets of reprogramming factors described herein are non-limiting and new sets of reprogramming factors may be discovered and new combinations of the individual factors described herein may be found to accomplish direct reprogramming of fibroblasts, myofibroblasts, or cardiac fibroblasts to cardiomyocytes. In some embodiments, the cardiac fibroblast-specific enhancers of the disclosure are operably linked to one or more genes or nucleotide sequences encoding a product that is a reprogramming factor. In some embodiments, the reprogramming factor is a polypeptide. In some embodiments, the reprogramming factor is a non-coding polynucleotide such as, for example, a microRNA (miRNA or mIR).

### Compositions and Kits

The disclosure provides compositions comprising polynucleotides and vectors comprising the enhancer sequences described herein. In some embodiments, the compositions comprise a polynucleotide comprising the enhancer sequences operably linked to a transgene. In some embodiments, the compositions comprise a vector comprising the enhancer sequences operably linked to a transgene. In some embodiments, the composition comprises pharmaceutically acceptable carriers, diluents, and excipients. In some embodiments, the composition comprises a pharmaceutically acceptable formulation.

The disclosure provides kits comprising polynucleotides and vectors comprising the enhancer sequences described herein. In some embodiments, the kit comprises a composition described herein.

### Methods of Use

The disclosure provides methods of use for polynucleotides and vectors comprising the enhancer sequences described herein. In some embodiments, the methods comprise providing polynucleotides and/or vectors comprising the enhancer sequences, contacting a host cell with the polynucleotides and/or vectors, and expressing transgenes in the host cell. Expression of the transgene in a host cell can be cell-type specific. In some embodiments, the method comprises expressing a transgene or nucleotide sequence encoding a product in a cardiac cell. In some embodiments, the method comprises expression of a transgene or nucleotide sequence encoding a product in a cardiac fibroblast. In some embodiments, the method comprises expressing a transgene or nucleotide sequence encoding a product in a stem cell. In some embodiments, the method comprises expressing a transgene or nucleotide sequence encoding a product in an induced pluripotent stem cell.

In some embodiments, the methods comprise providing a purified and/or isolated polynucleotide or vector comprising the enhancer sequences described herein. Providing the purified and/or isolated polynucleotide or vector can include, for example, the recombinant manipulation of the polynucleotides and vectors. Manipulation can include, for example, recombinant techniques for operably linking an enhancer described herein to a transgene or nucleotide sequence encoding a product, adding or removing nucleotide sequence encoding elements, such as, without limitation, purification tags, reporter genes, and/or TREs. In some embodiments, providing a purified and/or isolated polynucleotide or vector comprises purifying and/or isolating polynucleotides and vectors comprising the enhancer sequences described herein. Manipulation, isolation, and/or purification of polynucleotides or vectors can be achieved using any technique known in the art to accomplish the, such as those described in in Sambrook et al. Molecular Cloning: A Laboratory Manual; Cold Spring Harbor Laboratory Press, 4th edition 2012.

In some embodiments, the polynucleotides or vectors are provided as a pharmaceutically acceptable formulation.

In some embodiments, the method comprises contacting a host cell with a polynucleotide comprising an enhancer sequence described herein. In some embodiments, the method comprises contacting a host cell with a vector comprising an enhancer sequence described herein. It is understood that contacting the host cell with a polynucleotide or vector described herein results in the transmission of the enhancer sequence and, optionally, operably linked elements, e.g. a transgene, into the host cell. A “host cell” refers to a living cell into which a heterologous polynucleotide sequence, e.g. the enhancer sequences described herein, is to be or has been introduced. The living cell includes both a cultured cell and a cell within a living organism. Means for introducing the heterologous polynucleotide sequence into the cell are well known, e.g., transfection, electroporation, calcium phosphate precipitation, microinjection, transformation, viral infection, and/or the like. In some embodiments, transmission of the enhancer sequence and operably linked elements is achieved by transfection. In some embodiments, transmission of the enhancer sequence and operably linked elements is achieved by transduction. Transduction, as described herein, refers to the transmission of genetic material by a viral vector, e.g. an AAV vector comprising the enhancer sequences described herein. In some embodiments, the host cell is a cardiac cell. In some embodiments, the host cell is a cardiac fibroblast. In some embodiments, the host cell is an induced pluripotent stem cell. In some embodiments, the method provides a host cell comprising a polynucleotide or vector comprising the enhancers described herein.

In some embodiments, the methods comprise culturing a cell comprising the polynucleotide or vectors described herein. In some embodiments, culturing the cell comprising the polynucleotide or vector described herein results in the expression of a transgene operably linked to the enhancers described herein. A transgene expressed in a cell comprising the polynucleotide or vectors described herein will differ in expression level depending on the cell type comprising the polynucleotide or vector. For example, the transgene will be expressed in a cardiac cell but not, for example, a hepatocyte. For example, the transgene will be expressed at a higher level in a cardiac fibroblast than a cardiomyocyte.

In some embodiments, the method comprises delivering the polynucleotide or vector comprising the enhancers described herein to a cell, tissue, organ, or systemically within a living organism. The polynucleotide or vector can be delivered to the a living organism using any method known in the art. For example, delivery of the polynucleotide or vector can be delivered, without limitation, intravenously, intramuscularly, and subcutaneously. In some embodiments, the polynucleotide or vector is delivered to an organism in a host cell. For example, the polynucleotide or vector can be transfected or transduced into a cell ex vivo and the cell can be delivered to the living organism. In some embodiments, the polynucleotide or vector can be administered to a living organism as a pharmaceutical composition.

In some embodiments, the method comprises measuring the expression level by a host cell of a transgene operably linked to the enhancers described herein. The expression of a transgene can be determined in by any appropriate method known in the art. The transgene expression level can be determined by measuring an RNA product of the transgene. The transgene expression level can be determined by measuring the translation product, e.g. a polypeptide, of the transgene. The transgene expression level can be determined by measuring the level of product secreted by the cell into the cell culture media. In some embodiments, the expression level and/or rate is at least two, at least three, at least four, at least five, at least six, at least seven, at least eight, at least nine, or at least ten times greater in cardiac fibroblasts than cardiomyocytes.

### EXAMPLES

### Example 1: Identification of Putative Enhancer Sequences

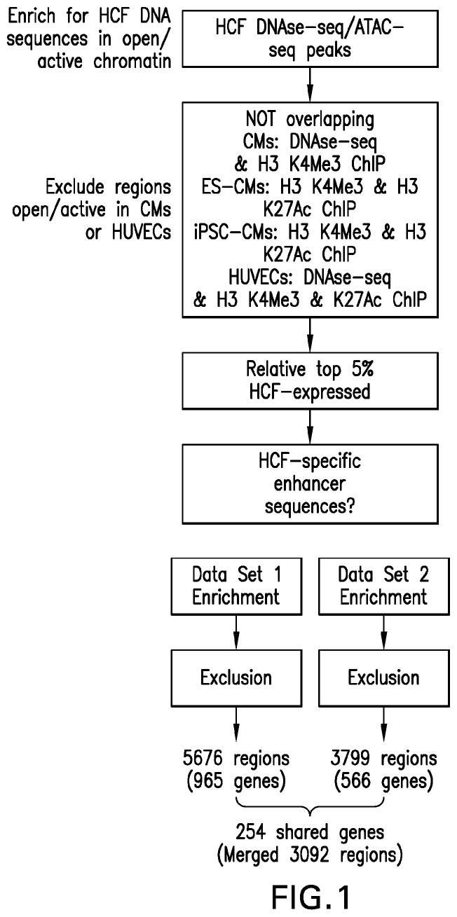

A computational screen was used to enrich for sequences of open chromatin near genes highly expressed in human cardiac fibroblasts (HCFs), but which did not bear the signature of open and active chromatin in cardiomyocytes (CMs) or endothelial cells (human umbilical vein endothelial cells, HUVECs). An informatics pipeline to identify highly expressed sequences was developed and performed on two independent complete HCF data sets (The ENCODE Project Consortium. Nature 489:57-74 (2012); Jonsson et al. JACC Basic Transl Sci. 30:590-602 (2016)) (FIG. 1). Specifically, for each data set, open chromatin in HCFs was identified based on DNase-seq or ATAC-seq peaks. Next, all regions were removed which overlapped with open chromatin or euchromatic histone modifications (H3K4Me3, H3K27Ac chromatin immunoprecipitation (ChIP)) in CMs (mature, embryonic stem cell-derived (ES-CMs), and induced pluripotent stem cell-derived CMs (iPSC-CMs) or HUVECs. Using the matched transcriptional analysis, regions upstream or within the introns of the most highly expressed genes (top 5%) in HCFs were selected. Only the intersection of the two independent data sets was considered, resulting in 3092 regions from 254 shared genes (FIG. 1). These 3092 computationally-derived regions were selected for experimental validation.

### Example 2: Identifying Enhancer Sequences with High Expression in Cardiac Fibroblasts and Weak Expression in Cardiomyocytes

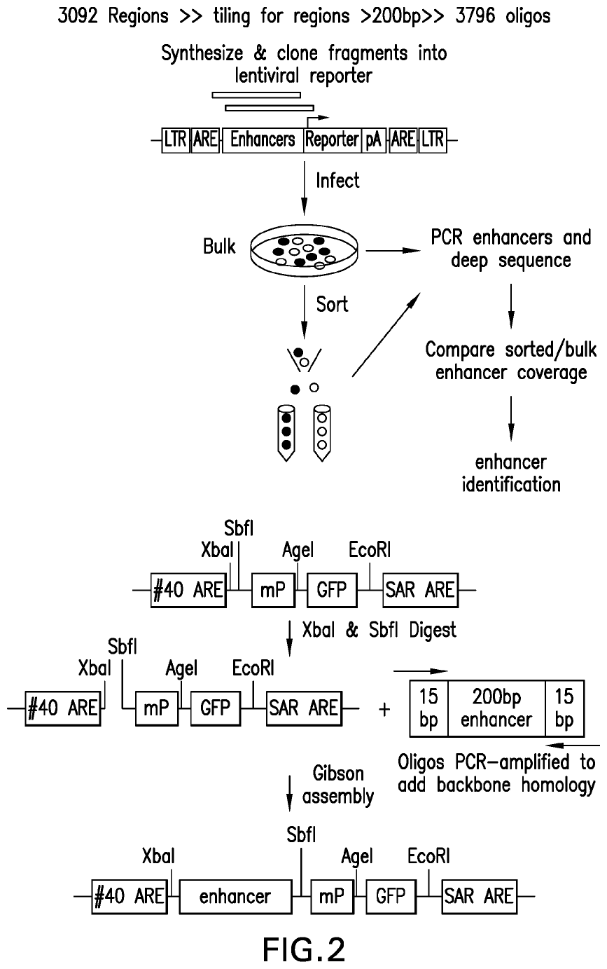

To screen the putative enhancer elements in vitro, a library of 3796 oligos was synthesized (tiling was implemented for regions >200 bp) and cloned upstream of a GFP reporter lentiviral vector (FIG. 2). Upon lentiviral generation, two independently derived HCF cell lines were transduced, using four million cells/replicate to allow for 1000X representation. These regions were then screened by fluorescence-activated cell sorting (FACS) expression analysis. Cells of interest based on GFP intensity were sorted and matched unsorted cells were collected. Sorts of transduced HCFs were performed in duplicate or triplicate for each HCF cell line. Further, to simultaneously exclude regions which promoted expression in iPSC-CMs, GFP-negative cells were sorted from transduced iPSC-CMs so that the presence of the region in the negative population could be confirmed.

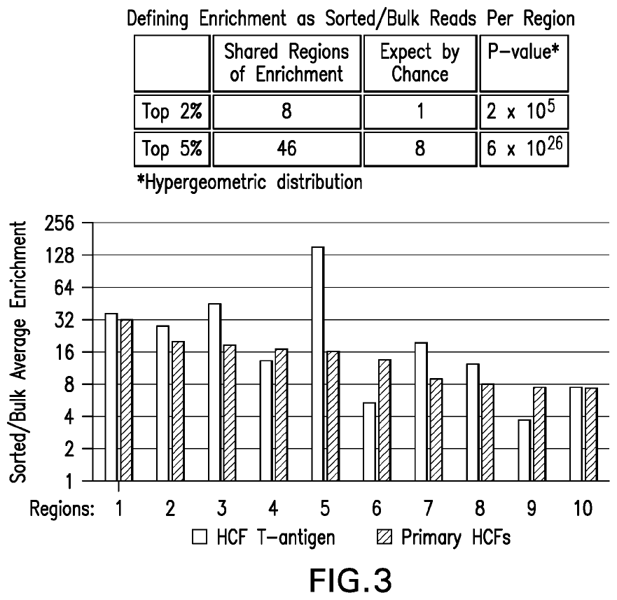

Following DNA isolation and amplification of putative enhancers, deep sequencing was performed to compare the representation of each element in sorted cells compared to the bulk unsorted cells. Enrichment was defined as the ratio of sorted/unsorted reads per region. Despite the two HCF lines coming from two different sources and one being T-antigen transformed, significant overlap in recovered enriched regions was observed (FIG. 3). Looking at the top 98th percentile of enrichment for each HCF cell type, 8 shared regions were observed, when only one is expected by chance. Looking at the top 95th percentile, 46 regions were shared, or 6-fold higher than anticipated by chance (FIG. 3). The top 16 shared regions were selected for validation (Table 1), the majority of which neighbored genes required for HCFs' primary function of extracellular matrix (ECM) deposition (collagen synthesis (COL6A1, COL4A1, PLOD2 and ADAMTS2) and generation of other ECM components (UGDH and FBLN1)). Other adjacent genes included those involved in TGF-β signaling (LTBP2) and cell migration (RAB31, CAPZB, RHOA). Although only the top 16 shared regions, i.e. putative enhancers, were selected for validation it should be acknowledged that other regions from the screen will also show cardiac cell and/or cardiac fibroblast selective expression.

TABLE 1
Top 16 Shared Regions of Enrichment

| | Enrichment | | | | |
|---|---|---|---|---|---|
| Genomic | | | Size | Neighboring | |
| Location | T-antigen | Primary | (bp) | Gene | |
| chr21: 47412473- | 36.67 | 32 | 73 | COL6A1 | intron |
| 47412546 | | | | | |
| chr18: 9726368- | 54 | 20.5 | 73 | RAB31 | intron |
| 9726441 | | | | | |
| chr4: 39504115- | 44.67 | 18.5 | 150 | UGDH | |
| 39504265 | | | | | |
| chr1: 19698103- | 152.2 | 16.29 | 73 | CAPZB | intron |
| 19698176 | | | | | |
| chr5: 178661375- | 19.62 | 9.03 | 88 | ADAMTS2 | intron |
| 178661463 | | | | | |
| chr3: 49425177- | 27.67 | 20 | 280 | RHOA | intron |
| 49425457 | | | | | |
| chr2: 242178535- | 13.33 | 17 | 73 | HDLBP | |
| 242178608 | | | | | |
| chr6: 169634154- | 8.41 | 7.7 | 73 | THBS2 | intron |
| 169634227 | | | | | |
| chr6: 169638903- | 7.54 | 7.35 | 223 | THBS2 | intron |
| 169639126 | | | | | |
| chr14: 102514441- | 9.93 | 5.72 | 73 | DYNC1H1 | |
| 102514514 | | | | | |
| chr3: 145817479- | 5.33 | 13.5 | 73 | PLOD2 | intron |
| 145817552 | | | | | |
| chr14: 92397863- | 3.7 | 7.57 | 479 | FBLN5 | intron |
| 92398342 | | | | | |
| chr14: 92369482- | 6.64 | 6.13 | 507 | FBLN5 | intron |
| 92369989 | | | | | |
| chr13: 110834336- | 6.08 | 5.64 | 205 | COL4A1 | intron |
| 110834541 | | | | | |
| chr14: 75071626- | 4.18 | 5.86 | 206 | LTBP2 | intron |
| 75071832 | | | | | |
| chr6: 169648019- | 6.83 | 3.87 | 82 | THBS2 | intron |
| 169648101 | | | | | |

### Example 3: Determining Strength and Selectivity of Enhancer Sequences

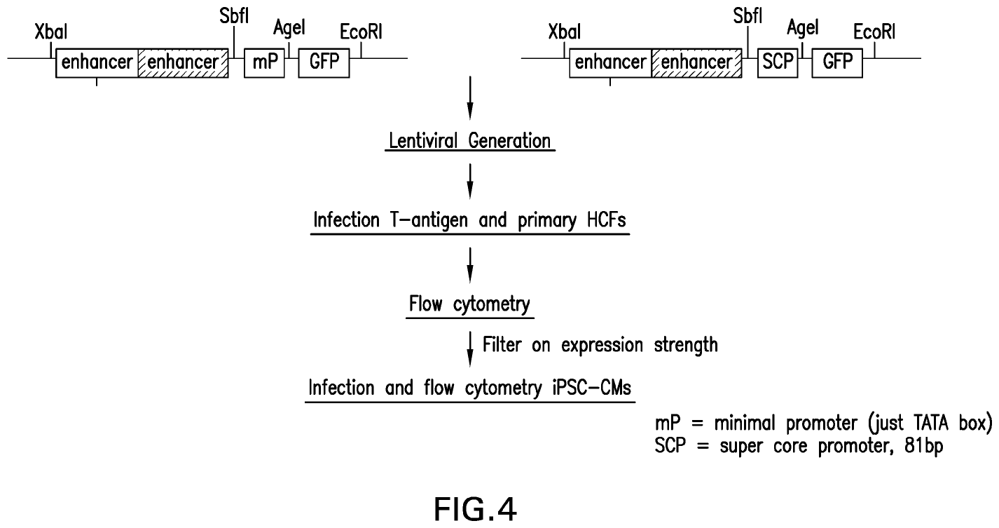

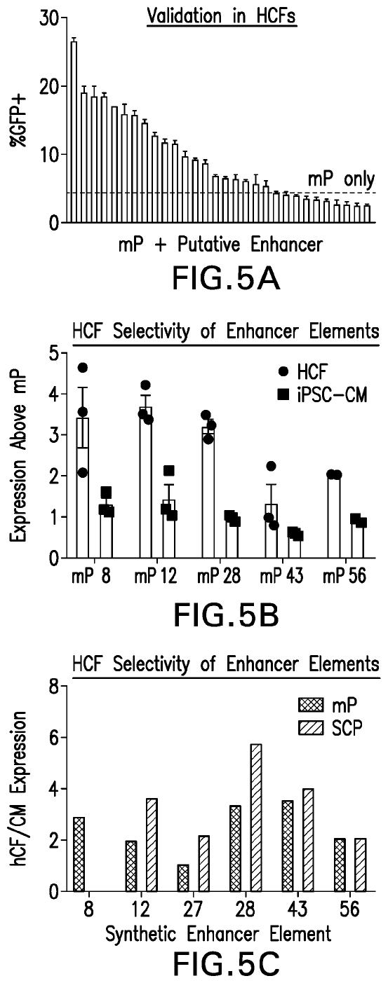

Given that more than half of the top shared regions were smaller than 100 base pairs, regions were combined for the secondary screen and tested for expression strength in HCFs compared to the construct with only a TATA box driving expression (minimal promoter, mP) (FIG. 4). For the top-expressing clones in HCFs, side-by-side lentiviral transduction of HCFs and human iPSC-CMs was performed to compare intensities of GFP expression (FIG. 5). Secondary element #8 from the intron of LTBP2 exhibited the greatest expression in HCFs and 3-fold selectivity (FIG. 5). Additionally, to further boost expression synthetic enhancers were cloned upstream the super core promoter (SCP) (Juven-Gershon et al. Nat Methods. 3:917-922 (2006)) and similarly tested. Cloned upstream of the SCP, synthetic enhancers retained two-sixfold selectivity for HCFs (FIG. 5). Given the synthetic selective enhancers (Table 2) range from 146-238 bp, additional elements can be added to boost expression or tune selectivity.

TABLE 2
Synthetic Selective Enhancers

| | SEQ ID | Size | Neighboring |
|---|---|---|---|
| ID | NO | (bp) | Genes |
| #12 | 17 | 146 | COL6A1-HDLBP |
| #27 | 18 | 238 | UGDH-ADAMTS2 |
| #28 | 19 | 223 | UGDH-HDLBP |
| #43 | 20 | 161 | CAPBZ-ADAMTS2 |
| #56 | 21 | 161 | COL6A1-ADAMTS2 |

What is claimed is:

1. A recombinant polynucleotide, comprising a cardiac cell-specific enhancer that shares at least 80% identity to any one of SEQ ID NOs: 1-21.

2. The polynucleotide of claim 1, wherein the polynucleotide comprises a sequence encoding a transgene product operatively linked to the enhancer.

3. The polynucleotide of claim 1 or 2, wherein the enhancer is a cardiac fibroblast-specific enhancer.

4. The polynucleotide of claim 2 or 3, wherein the polynucleotide expresses the transgene product at a level and/or rate of at least two, at least three, at least four, at least five, at least six, at least seven, at least eight, at least nine, or at least ten times greater in cardiac fibroblasts than cardiomyocytes.

5. The polynucleotide of claim 4, wherein the polynucleotide expresses the transgene product at a level and/or rate of at least two times greater in cardiac fibroblasts than cardiomyocytes.

6. The polynucleotide of claims 1 to 5, wherein the polynucleotide comprises a sequence that shares at least 80%, at least 85%, at least 90%, at least 95%, at least 98%, or 100% identity to SEQ ID NOs: 1-21.

7. The polynucleotide of claims 1 to 5, wherein the polynucleotide comprises a sequence that shares at least 80%, at least 85%, at least 90%, at least 95%, at least 98%, or 100% identity to at least two sequences selected from SEQ ID NOs: 1-21.

8. The polynucleotide of claims 1 to 5, wherein the polynucleotide comprises a sequence that shares at least 80%, at least 85%, at least 90%, at least 95%, at least 98%, or 100% identity to SEQ ID NOs: 19.

9. The polynucleotide of claims 1 to 5, wherein the polynucleotide comprises a sequence that shares at least 80%, at least 85%, at least 90%, at least 95%, at least 98%, or 100% identity to SEQ ID NOs: 20.

10. The polynucleotide of claims 1 to 5, wherein the polynucleotide comprises a sequence that shares at least 80%, at least 85%, at least 90%, at least 95%, at least 98%, or 100% identity to SEQ ID NOs: 21.

11. A vector comprising the polynucleotide of any one of claims 1 to 10.

12. The vector of claim 11, wherein the vector is a viral vector.

13. The vector of claim 12, wherein the viral vector is a lentivirus, a retrovirus, an adenovirus, an adeno-associated virus, or a hybrid virus.

14. The vector of claim 11, wherein the vector is a plasmid.

15. The vector of claim 11, wherein the vector is an artificial chromosome.

16. An isolated cell comprising the polynucleotide of any one of claims 1 to 10.

17. An isolated cell comprising the vector of any one of claims 11 to 15.

18. The cell of claim 16, wherein the cell is a cardiac cell.

19. The cell of claim 18, wherein the cardiac cell is a cardiac fibroblast and/or cardiomyocyte.

20. The cell of claim 16, wherein the cell is a stem cell.

21. The cell of claim 16, wherein the cell is an induced pluripotent stem cell.

22. A pharmaceutical composition comprising the cell of any one of claims 16 to 21.

23. A method of expressing a transgene product, comprising introducing a polynucleotide into a cell, wherein the polynucleotide comprises a cardiac cell-specific enhancer that shares at least 80%, at least 85%, at least 90%, at least 95%, at least 98%, or 100% identity to any one of SEQ ID NOs: 1-21.

24. The method of claim 23, wherein the polynucleotide comprises a sequence encoding a transgene product operably linked to the enhancer.

25. The method of claim 23 or 24, wherein the polynucleotide is introduced into the cell using a viral vector.

26. The method of claim 23, wherein the cell is a cardiac cell.

27. The method of claim 25, wherein the cardiac cell is a cardiac fibroblast and/or cardiomyocyte.

28. The method of claim 23, wherein the cell is a stem cell.

29. The method of claim 28, wherein the cell is an induced pluripotent stem cell.

30. The method of any one of claims 23 to 29, wherein the transgene product is expressed at a level and/or rate of at least two, at least three, at least four, at least five, at least six, at least seven, at least eight, at least nine, or at least ten times greater in cardiac fibroblasts than cardiomyocytes.


SEQUENCE LISTING (21 sequences)

SEQ ID NO: 1 - DNA, 73 bp, Homo sapiens
ctcagagcgggtgggacctggggaaccaggagattccggcctctgcaaccgtggggcatgcggtggaggggtg

SEQ ID NO: 2 - DNA, 73 bp, Homo sapiens
cagatgagccctggcagcaggtggacttgggcacgtattcgctgtggcaaatcatgtaatctctggggctcac

SEQ ID NO: 3 - DNA, 150 bp, Homo sapiens
aaacactgctgcttttctgcttgcttgggcactacattgacaacaaccccctggcatatttttttattctccctaacttactccacctccccatcctcctatccccatatacatactgctctgaggaaatggaaaatagctccttattaa

SEQ ID NO: 4 - DNA, 73 bp, Homo sapiens
gacgccatgggtgagggtcagaggcttttggtgggaggtggtggtgccttctgcctgggaagccccccagcac

SEQ ID NO: 5 - DNA, 88 bp, Homo sapiens
cccagtccaccgtgccctggacgctgctcatccactaggactgggcttccgtgtgcagggcttcaccctcgctctcctctctgcctgc

SEQ ID NO: 6 - DNA, 281 bp, Homo sapiens
aaaaaaaaacaaaaaaaaaaaccctattatgtttgttttctatgcttttatgatgtttttgcatacatttcttttagatgtgtgtatcttttttctgtacacaccagaggaagaaataataacaatgtcacatatgtttgagtcactctgaaatcatcaactcttggttctttggttctagcatgtcttacacctgtttactcttactgttttaatgtctgtttactcattacagatgatgtcaaggaaggggatggctttctctttttttttttttcctg

SEQ ID NO: 7 - DNA, 73 bp, Homo sapiens
gccctggcaggggagtggcctggaggggacactctctaaagactgcttctaaagaccacgtcttctcatgact

SEQ ID NO: 8 - DNA, 73 bp, Homo sapiens
cccatgcaccatgttccacaccgcacaactacctacacaccatgtttcacaacacacagctactcacatgcca

SEQ ID NO: 9 - DNA, 224 bp, Homo sapiens
gggacattctgggaatgtttgggggatggtgactagtttagtttgggagaagcgttgcctgatcttagtgcagcagcaacttggggtgtagacctgaagcaccttgccaacggcacggcactggggagccacggatggctcaggagctctgggtggggtcagcagagctaacttgggaagggaactcggggcaggggtgggaggcagcacagagacctgagggc

SEQ ID NO: 10 - DNA, 73 bp, Homo sapiens
gaccattggttccactgtgccggacggaacgtactcacaccggtgtcacctcagagcaggtgtccagctgagc

SEQ ID NO: 11 - DNA, 73 bp, Homo sapiens
aaatttgattaaaatattatattaccaaggaggaacagaacactatgtagttatgggaaaagtggggttctac

SEQ ID NO: 12 - DNA, 480 bp, Homo sapiens
ttctcagatcaataacaataacaaaaatcctggactagaagagagaatcaagatattgcttgtaatttcacaaatggccggaatttctgcagtccccggagtagcagggctctagtaaaaggctgctttctcagccgcaaaatatttcacaaacatgagttactgatggagtctctacatttgccactgagccaagctggttagcaaagctagaacttggaatgtccacccagagagatgacaagttgagcccccaagggaatgagttgacagagttgagtggcggccacgttcagggacccggtcttagaggcagcgtgccatgagaggaaggttcctgaatgggagtccacatcttgggtgccccagccactcggcaccagctcaccccaccccctgaggcttcagcaccacaccatcctcagccccacaccgtccttctctgggatgtctatgtggccagctcagaactctacgtat

SEQ ID NO: 13 - DNA, 508 bp, Homo sapiens
ttcttctctctctaagataaagcctgctggttataacagagttgttatttttggagtcttattaaagactctagcacagtctagccaaaacagaatgcttttctggtgtacggacagagcctttctttaactccaccctgtagtagttgggtgcctatggctgcaatgccttggccagggcattgtgtgcttccagaaggttctcctcccttgatcactggtggggttttgatttcctatgaactcaattccctgactacccataaagagataacaaaaagtacaaaagatcccagtgaggagaaaggacaacatacttcctccaacacagagcatcccttttttaactgagtactcatgaacatagtttctacatgctgtggatcctgactcctgtaatctccccatctccccgagggctccttcttctcattccctctgagcctattcctcacccatcctgacctgtatcatagtctgtgaactccttgcccctcccacctgctcc

SEQ ID NO: 14 - DNA, 206 bp, Homo sapiens
catcttctgtgaggctgctgcacagatagggtctgtcctcctcactgccacaaagccaagcaactccatgtcacatgggtcacggcctcaggtgaaggaggcactgtgtgaggcaatggtggaatgtgagggagttgtgagtgtgacgaggcctcagcgcctggccctcctgaacagggatgctctgctgcccgcccagcctgagg

SEQ ID NO: 15 - protein, 207 aa, Homo sapiens
CACCCGGCCCTGACTCTGCAGCTAAAGACCCACATGCCTAGACCAGGGCGTCTCCCTAGAAGGGCTTCTCCTGGAGATCTCTAGGGACTGACTCCTTGGCAGGCTGGGCCGGGCTCTGGCTTCCATCCTTATCATTGGAAATCATCACCGGGCACTGGAACTAGAGGGTAAGAGGGGAGGGAGAAGTGCAGAGGCTGGACAGGCAAA

SEQ ID NO: 16 - DNA, 82 bp, Homo sapiens
atgaatggtgtttatgtgcgtctgcatgtgcgtgcccatgtatggtgtttgtgtgcacatgggcatgcatatgtgagggtgt

SEQ ID NO: 17 - DNA, 146 bp, Artificial Sequence
  Made in Lab - construct enhancer sequence
ctcagagcgggtgggacctggggaaccaggagattccggcctctgcaaccgtggggcatgcggtggaggggtggccctggcaggggagtggcctggaggggacactctctaaagactgcttctaaagaccacgtcttctcatgact

SEQ ID NO: 18 - DNA, 238 bp, Artificial Sequence
  Made in Lab - construct enhancer sequence
aaacactgctgcttttctgcttgcttgggcactacattgacaacaaccccctggcatatttttttattctccctaacttactccacctccccatcctcctatccccatatacatactgctctgaggaaatggaaaatagctccttattaacccagtccaccgtgccctggacgctgctcatccactaggactgggcttccgtgtgcagggcttcaccctcgctctcctctctgcctgc

SEQ ID NO: 19 - DNA, 223 bp, Artificial Sequence
  Made in Lab - construct enhancer sequence
aaacactgctgcttttctgcttgcttgggcactacattgacaacaaccccctggcatatttttttattctccctaacttactccacctccccatcctcctatccccatatacatactgctctgaggaaatggaaaatagctccttattaagccctggcaggggagtggcctggaggggacactctctaaagactgcttctaaagaccacgtcttctcatgact

SEQ ID NO: 20 - DNA, 161 bp, Artificial Sequence
  Made in Lab - construct enhancer sequence
gacgccatgggtgagggtcagaggcttttggtgggaggtggtggtgccttctgcctgggaagccccccagcaccccagtccaccgtgccctggacgctgctcatccactaggactgggcttccgtgtgcagggcttcaccctcgctctcctctctgcctgc

SEQ ID NO: 21 - DNA, 161 bp, Artificial Sequence
  Made in Lab - construct enhancer sequence
ctcagagcgggtgggacctggggaaccaggagattccggcctctgcaaccgtggggcatgcggtggaggggtgcccagtccaccgtgccctggacgctgctcatccactaggactgggcttccgtgtgcagggcttcaccctcgctctcctctctgcctgc